(19) United States
(12) Patent Application Publication
(10) Pub. No.: US 2009/0094721 A1
(43) Pub. Date: Apr. 16, 2009

(54) AUTOMATED SENSITIVITY SETTING FOR AN AUTO-DARKENING LENS IN A WELDING HELMET

(75) Inventor: William J. Becker, Manitowoc, WI (US)

Correspondence Address: FLETCHER YODER (ILLINOIS TOOL WORKS INC.), P.O. BOX 692289, HOUSTON, TX, 77269-2289, US

(73) Assignee: ILLINOIS TOOL WORKS INC., Glenview, IL (US)

(21) Appl. No.: 12/245,522

(22) Filed: Oct. 3, 2008

Related U.S. Application Data
(60) Provisional application No. 60/979,254, filed on Oct. 11, 2007.

(51) Int. Cl. A61F 9/06

(52) U.S. Cl. 2/8.8; 250/205; 349/14

(57) ABSTRACT

There are provided systems and methods for automatic adjustment of sensitivity threshold settings in an auto-darkening lens. For example, a user may initiate an automatic sensitivity setting process by activating a user interface (e.g., a button, a switch, a slider, etc.). The automatic sensitivity setting process may proceed to gradually increase the sensitivity threshold voltage until it exceeds an optical voltage based on the ambient light. The process may then be terminated. In some embodiments, a hysteresis may be added to the sensitivity threshold voltage to prevent lens flicker. The automatic sensitivity setting process may be implemented in a digital or analog system. These automatic sensitivity setting processes and systems may save time and provide for more precise sensitivity adjustment.

### CROSS REFERENCE TO RELATED APPLICATION

This application is a Non-Provisional Patent Application of U.S. Provisional Patent Application No. 60/979,254, entitled “AUTOMATED SENSITIVITY SETTING FOR AN AUTO-DARKENING LENS IN A WELDING HELMET,” filed Oct. 11, 2007, which is herein incorporated by reference in its entirety.

### BACKGROUND

The invention relates generally to welding helmets, and more particularly to a welding helmet having automated sensitivity settings for an auto-darkening lens.

Welding operations are generally performed with certain precautions due to the potential exposure of the welding operator to high heat, flames, weld spatter and ultraviolet light. For example, welders may wear goggles and/or helmets for protection. These helmets generally include a face plate (or lens) that is darkened to prevent or limit exposure to the arc light. In some helmets, the lens is constantly dark with the user flipping down the helmet during welding. In other helmets, the lens may change from a clear state to a darkened state.

### BRIEF DESCRIPTION

In accordance with embodiments of the present technique, there is provided a welding helmet having an auto-darkening lens and an automatically-adjusting sensitivity setting. In one embodiment, the welding helmet includes an auto-darkening lens having optical sensors for sensing optical energy, optical sensing circuitry for converting the sensed optical energy into an electrical voltage, sensitivity circuitry for automatically adjusting a threshold sensitivity voltage based on the electrical voltage, and a user input to initiate automatic adjustment of the threshold sensitivity voltage.

In accordance with another embodiment, there is provided a method for automatically adjusting sensitivity in an auto-darkening lens, including detecting ambient light intensity, converting the ambient light intensity to an optical voltage, comparing the optical voltage to a sensitivity voltage and outputting a digital signal based on the comparison; and automatically adjusting the sensitivity voltage stepwise until the digital signal changes value.

In accordance with a further embodiment, there is provided an auto-darkening lens, including a user interface for initiating an automatic sensitivity setting process, sensitivity setting circuitry setting a sensitivity voltage to an initial value upon initiation of the automatic sensitivity setting process, optical sensors for detecting light intensity, an optical sensing circuit for converting the light intensity to an optical voltage, and a comparator for comparing the sensitivity voltage to the optical voltage and outputting a digital signal indicative of the comparison. The sensitivity setting circuitry may alter the sensitivity voltage until the digital signal from the comparator changes.

### DRAWINGS

These and other features, aspects, and advantages of the present invention will become better understood when the following detailed description is read with reference to the accompanying drawings in which like characters represent like parts throughout the drawings, wherein:

FIG. 1 is an illustration of an exemplary arc welding system including a welding helmet in accordance with embodiments of the present technique;

FIG. 2 is an illustration of an exemplary embodiment of the welding helmet of FIG. 1 including an automatic sensitivity adjustment;

FIG. 3 is a diagrammatical illustration of an exemplary embodiment of the welding helmet of FIG. 2;

FIG. 4 is a graphical illustration of an exemplary embodiment of an automatic sensitivity adjustment sequence;

FIG. 5 is a block diagram of an exemplary embodiment of a digital automatic sensitivity adjustment system;

FIG. 6 is a flow chart of an exemplary embodiment of processor logic of the digital automatic sensitivity adjustment system of FIG. 5;

FIG. 7 is a block diagram of an exemplary embodiment of an analog automatic sensitivity adjustment system in accordance with aspects of the present technique;

FIG. 8 is a schematic diagram of an exemplary embodiment of the analog automatic sensitivity adjustment system of FIG. 7; and

FIG. 9 is a timing diagram of an exemplary embodiment of the analog automatic sensitivity adjustment system of FIG. 7.

### DETAILED DESCRIPTION

One or more specific embodiments of the present invention will be described below. These described embodiments are only exemplary of the present invention. Additionally, in an effort to provide a concise description of these exemplary embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Auto-darkening welding lenses detect when a weld has been struck, for example, by employing an optical sensor. Upon detection of a weld, the lens is darkened to a predetermined shade, thereby protecting the user's eyes from the bright light emitted from the welding arc. Embodiments of the present technique provide for automatically adjusting the sensitivity to an optimized value in a given ambient lighting environment. The systems and methods herein may generally be applied to an auto-darkening lens that darkens based on the intensity of light detected by optical sensors, as described below.

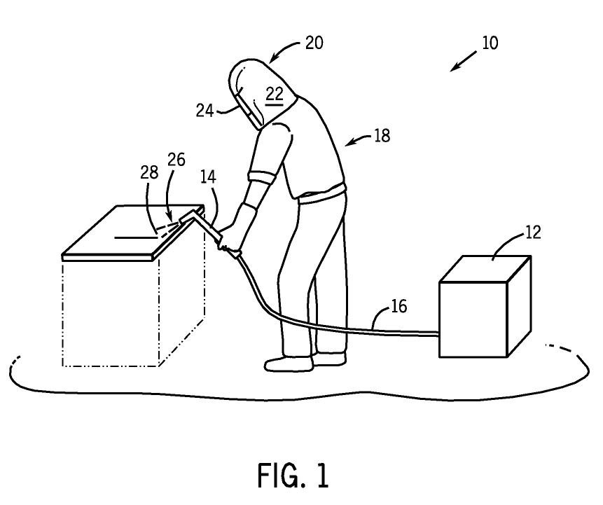

Embodiments of the present invention may have uses in a variety of welding applications. For example, FIG. 1 illustrates an arc welding system 10. As depicted, the arc welding system 10 may include a power supply 12 that generates and supplies a current to an electrode 14 via a conduit 16. In the arc welding system 10, a direct current (DC) or alternating current (AC) may be used along with the consumable or non-consumable electrode 14 to deliver the current to the point of welding. In such a welding system 10, an operator 18 may control the location and operation of the electrode 14 by positioning the electrode 14 and triggering the starting and stopping of the current flow.

In welding operations employing the exemplary welding system 10 depicted in FIG. 1, welding is generally performed with certain precautions due to the generation of heat and bright light in the visible and non-visible spectra. To avoid overexposure to such light, a helmet assembly 20 is worn by the welding operator 18. The helmet assembly 20 includes a helmet shell 22 and a lens assembly 24 that may be darkened to prevent or limit exposure to the light generated by a welding arc 26, as discussed below.

When the operator 18 begins the welding operation by applying current from the power supply 12 to the electrode 14, the arc 26 is developed between the electrode 14 and a work piece 28. The electrode 14 and the conduit 16 thus deliver current and voltage sufficient to create the electric arc 26 between the electrode 14 and the work piece 28. The arc 26 melts the metal (the base material and any filler material added) at the point of welding between electrode 14 and the work piece 28, thereby providing a joint when the metal cools. The welding system 10 may be configured to form a weld joint by any known technique, including shielded metal arc welding (i.e., stick welding), metal inert gas welding (MIG), tungsten inert gas welding (TIG), gas welding (e.g., oxyacetylene welding), and/or resistance welding.

As described below, the exemplary helmet assembly 20 used in the welding system 10 includes the lens assembly 24 having the functionality to transition between a clear state and a darkened state. Generally, the lens assembly 24 that transitions between clear and dark states may include electronic components which cause the lens to automatically darken (e.g., an LCD that darkens when a voltage is applied across the layer) when sensors detect bright light that is in excess of a threshold value. For example, the operator 18 may “turn on” the lens assembly 24 to provide a voltage across the lens and associated electronic components, thereby causing the assembly 24 to transition from a light or relatively clear state to a darkened state.

In particular embodiments, the lens assembly 24 may include electronic components that cause the lens to automatically darken when sensors detect bright light that is in excess of a threshold value, for example, by triggering circuitry of the lens assembly 24 to provide a voltage across the lens. In addition to darkening the lens, the helmet assembly 20 may provide for automatic adjustment of the threshold value of sensed light that triggers the lens assembly 24 to transition between light and dark states. For example, the helmet assembly 20 may include a circuit designed to automatically adjust the light sensitivity to an optimized value based on the ambient light, thereby automatically setting the level of external light that triggers the transition of the lens assembly 24 between states.

The automatic sensitivity setting function of the helmet assembly 20 enables the operator 18 to set the threshold light limit more precisely than a manual adjustment would allow. For example, a manual sensitivity adjustment may entail holding a helmet with the lens facing the work area in which the user will be welding, and then gradually increasing the sensitivity until the lens darkens in the ambient light. The sensitivity setting is then decreased a very small amount such that the lens is in a clear state in the ambient light but converts to the darkened state in brighter light. This manual sensitivity adjustment process can be time-consuming and is often neglected, forgotten, or performed improperly. In particular, this can be an issue for users who change welding environments often. For example, when changing from brighter to darker environments, the sensitivity usually needs to be readjusted in order for the auto-darkening lens to function most effectively. In addition, in low-amperage welding process, such as some tungsten inert gas (TIG) welding, the arc may be only a little brighter than the ambient lighting. In these cases, it may be desirable to automatically set the sensitivity as close as possible to the point at which the lens darkens in ambient lighting. A very small change in light intensity will then trigger the lens to transition to the dark state.

In an exemplary embodiment of the present technique, a user may direct the auto-darkening lens toward the work area and trigger the automatic sensitivity setting, for example, by pressing a button. Circuitry in the lens may detect the intensity of ambient light in the work area and convert the intensity to an optical voltage. The optical voltage may then be compared to a sensitivity voltage which dictates at what light intensity the lens will darken. The sensitivity voltage may be changed (e.g., increased or decreased, depending on the system) stepwise until the sensitivity voltage passes the optical voltage (e.g., the sensitivity voltage is greater than or less than the optical voltage, depending on the system). When the sensitivity voltage is set, the helmet may notify the user that the sensitivity has been automatically set, for example, by “flashing” the lens between the light and dark states, illuminating an LED, displaying a message on a screen or the lens, emitting a sound, or otherwise indicating that the sensitivity setting process is complete.

In addition to the sensitivity threshold, a time delay for transitioning between the darkened and clear states may be set by the operator 18. Such a setting may govern the time delay between detecting that the arc is extinguished and transitioning the lens assembly 24 from its dark state to its clear state. Additionally, a shade control may facilitate operator adjustment of the darkness of the lens in the “dark” state. Certain of the settings of the welding helmet assembly 20 may be pre-set at the time of manufacture, and may be re-adjusted by the operator 18. In particular, functions of the helmet assembly 20 may have adjustable settings controlled by analog or digital knobs, sliders, switches, buttons, and so forth. Accordingly, to make adjustments to the settings, an operator 18 may adjust the settings prior to welding, and/or re-adjust the settings once welding has begun. In accordance with embodiments of the present technique, the light sensitivity settings may be automatically adjusted, for example, by the operator 18 pressing a button or giving an audible voice command.

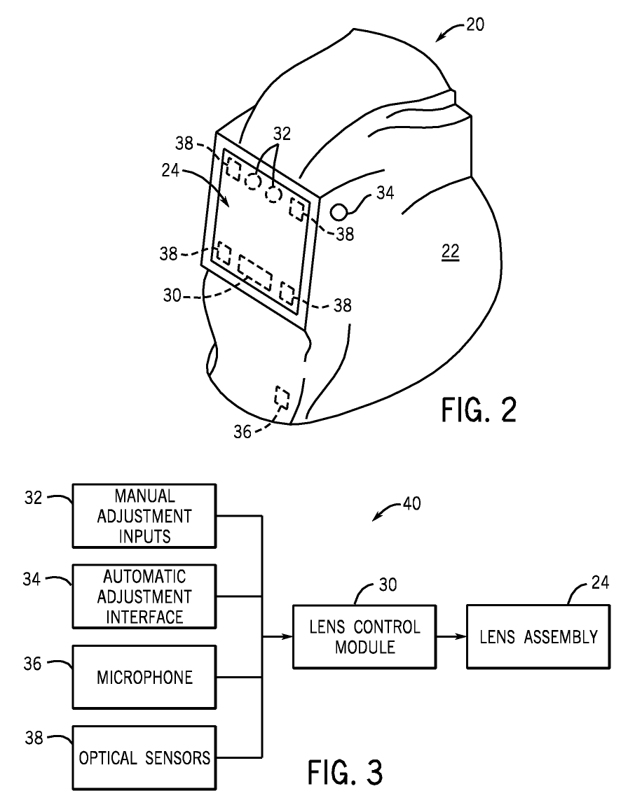

An exemplary embodiment of the helmet assembly 20 of FIG. 1 is illustrated in FIG. 2. The helmet shell 22 may constitute the general frame and support for the components of the welding helmet assembly 20. For example, the helmet shell 22 provides a partial enclosure about the face and neck of the operator 18 to shield the operator 18 from exposure to the high heat and bright light produced during welding. In addition to providing general protection, the helmet shell 22 provides a location to mount the lens assembly 24 and any additional accessories or control circuitry, such as a lens control module 30.

The lens control module 30 may include circuitry configured to monitor and control the state of the lens assembly 24, as well as circuitry to control other functions of the helmet assembly 20. In one embodiment, the lens control module 30 may be provided as a component of the lens assembly 24. For example, the lens assembly 24 may be mounted to the helmet shell 22 as a single unit. In another embodiment, the lens control module 30 may be a component that is separate from the lens assembly 24. For example, where the lens control module 30 is separate from the lens assembly 24, it may be mounted remotely in the helmet shell 22 with a connection (e.g., via wire conductors) to the lens assembly 24 sufficient to transmit control signals. As will be discussed in further detail, the lens control module 30 may acquire and process various inputs, compare the inputs to the values stored in a memory, and carry out programmed functionality to provide corresponding outputs to accessories related to the welding helmet assembly 20, particularly to lighten and darken the lens.

Exemplary inputs to the lens control module 30 may include user interface inputs and sensor inputs. For example, user interface inputs may include one or more manual adjustment inputs 32 and an automatic adjustment interface 34. The manual inputs 32 may include dials disposed inside or outside of the helmet shell 22 (e.g., coupled to the lens assembly 24) that provide signals when the dials are manipulated by the operator 18 (FIG. 1). By disposing the manual inputs 32 within the helmet shell 22, the operator 18 may be discouraged from adjusting sensitivity and other settings while the arc 26 is lit. The manual inputs 32 may take any form which provides a corresponding signal in response to the input of the operator 18. For example, the manual inputs 32 may include digital encoders, knobs, potentiometers, touch-sensitive sensors, buttons, keys, and so forth. The manual adjustment inputs 32 may enable the operator 18 (FIG. 1) to manually adjust helmet settings. For example, as described above, the operator 18 may adjust the light sensitivity threshold by directing the lens assembly 24 towards the work area and gradually increasing the sensitivity until the lens darkens, then dialing the threshold back a small amount. In addition, the operator 18 may change the time delay setting such that the lens assembly 24 transitions from the dark state to the clear state more quickly or slowly.

In accordance with embodiments of the present technique, the automatic adjustment interface 34 may initiate an automatic sensitivity-setting process which may override or replace the manual sensitivity settings. For example, the operator 18 may face the helmet assembly 20 towards the work area and activate the automatic adjustment sequence via the automatic adjustment interface 34. The lens assembly 24 may then automatically adjust the light sensitivity setting without further input from the operator 18, as described in more detail below. The automatic adjustment interface 34 may be, for example, a touch-sensitive sensor, a button, a key, a selectable menu, or any other user interface device which may initiate the automatic adjustment sequence. In some embodiments, the automatic adjustment interface 34 may be a dedicated button, while in other embodiments the interface 34 may be integrated into another feature of the helmet assembly 22. For example, the helmet assembly 22 may include a power button and/or a reset button which also acts as the automatic adjustment interface 34. That is, the automatic sensitivity adjustment sequence may be integrated into the power-up or start-up sequence.

Additionally, the automatic adjustment interface 34 may include a microphone 36 configured to pick up audible voice commands from the operator 18 so that settings to the lens assembly 24 may be adjusted hands-free. Audible commands may include adjusting the light sensitivity threshold and/or the time delay, directing the lens to switch to the dark or clear state, and so forth. In one embodiment, the automatic light sensitivity adjustment may be triggered by an audible voice command received through the microphone 36.

Further inputs to the lens control module 30 may include optical sensors 38, which may be photodetectors configured to sense light and/or electromagnetic sensors configured to detect electromagnetic emissions. The optical sensors 38 may determine the intensity of the light experienced at the lens and output a signal indicative of the light intensity to the lens control module 30. Based on the signal provided by the sensors 38, the lens control module 30 may output a signal to the lens assembly 24 to change to the light or dark state. The auto-darkening lens may operate by comparing the detected light intensity to the sensitivity threshold. That is, the optical sensors 38 may be connected to an amplification and/or voltage biasing circuit which outputs a signal (e.g., voltage) directly related to the intensity of light detected by the optical sensors 38. This voltage is then compared to a threshold voltage (e.g., the sensitivity voltage), and the result of the comparison determines if the lens state should be dark or light. The optical sensors 38 may also be utilized in the automatic sensitivity-setting process, as described below.

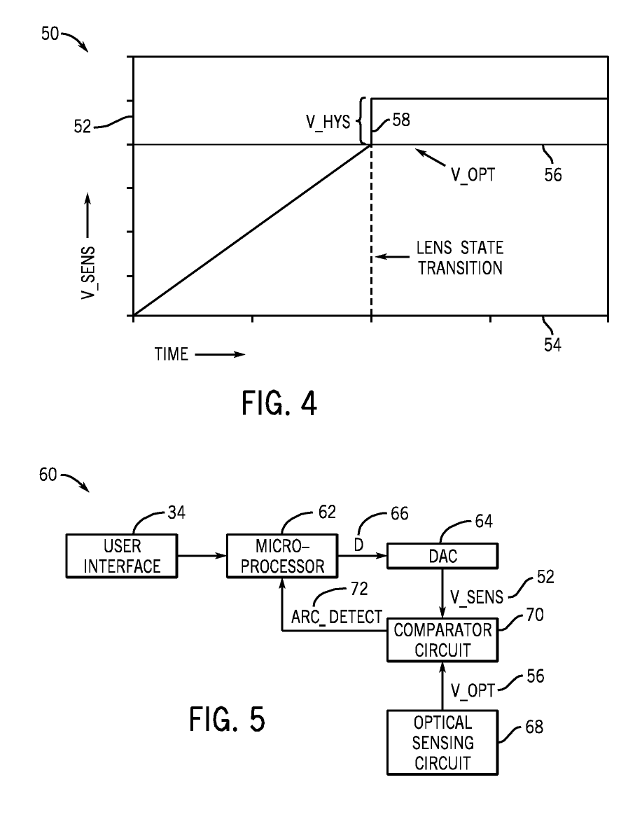

The signals provided by the various inputs 32-38 may be monitored by the lens control module 30, as illustrated by a control configuration 40 in FIG. 3. For example, in response to a signal from the optical sensors 38 indicating that the arc 26 has been lit (FIG. 1), the lens control module 30 may send a command to the lens assembly 24 to darken the lens. In another embodiment, a signal from the automatic adjustment interface 34 may initialize the automatic sensitivity adjustment. In addition, the lens control module 30 may be configured to give priority to one input over another. For example, to ensure that the lens is darkened when the arc 26 is present, the lens control module 30 may send a command to the lens assembly 24 to darken the lens even if the last audible command to the microphone 36 was to clear the lens. Similarly, to prevent inadvertent clearing of the lens during welding, the lens control module 30 may not respond to command signals to clear the lens while the optical sensors 38 detect the arc 26.

The automatic sensitivity-setting process may also be suspended while the optical sensors 38 detect the arc 26 (FIG. 1), even if the automatic adjustment interface 34 is triggered. This feature may prevent the sensitivity settings from being adjusted based on the bright arc light instead of the ambient light. In some embodiments, it may be desirable to override the optical sensors 38. For example, if the sensitivity threshold is too low, the optical sensors 38 may interpret ambient lighting as the arc 26. In these cases, override functionality may be provided, for example, by triggering the automatic adjustment interface 34 multiple times or for a longer duration, or by providing an audible command to override the optical sensor input 38. The automatic adjustment interface 34 may also override the manual inputs 32. That is, if the operator adjusts sensitivity settings manually and subsequently triggers the automatic adjustment interface 34, the automatic sensitivity-setting process may take precedence over the manual settings. In some embodiments, an additional input may be provided to serve as a switch between the manual and automatic sensitivity inputs 32 and 34 such that the input method must be selected before the sensitivity settings are changed. After the sensitivity has been adjusted via the automatic adjustment interface 34, changes to the manual inputs 32 may be applied to the pre-automatic settings (i.e., the sensitivity threshold may be adjusted from the previous manual adjustment point) or to the current settings (i.e., the sensitivity threshold may be adjusted from the automatically set point).

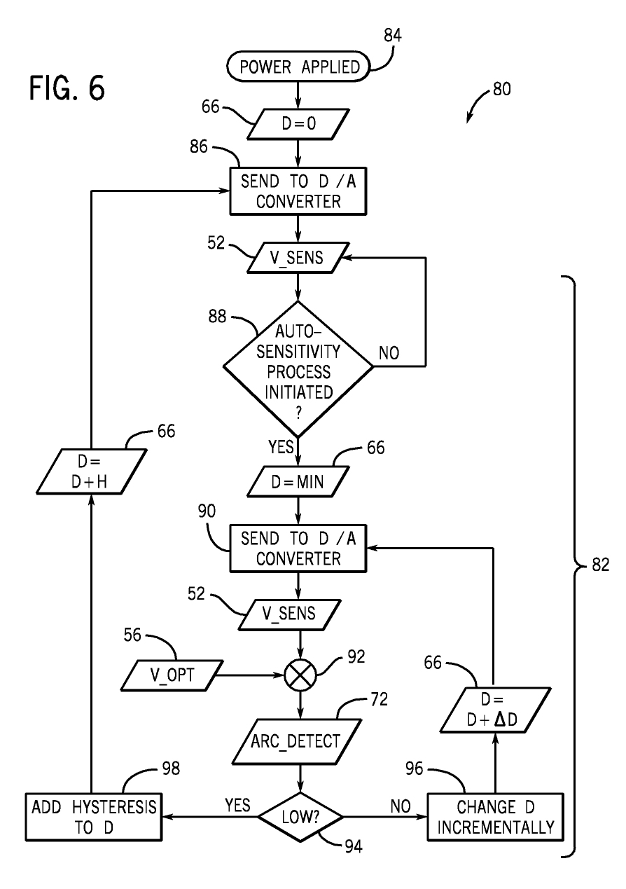

Turning to FIG. 4, a plot graphically illustrates an exemplary embodiment of an automatic sensitivity adjustment sequence 50. In the illustrated embodiment, the sensitivity threshold may be adjusted using a digital or an analog method. In either method, a sensitivity threshold voltage (V_SENS) 52 is adjusted over a time 54. Upon initiation of the automatic sensitivity adjustment sequence 50, the sensitivity voltage 52 is set to zero, thereby setting the lens to the dark state (e.g., the sensitivity is so low that the arc detection circuitry interprets any amount of detected light as arc light). The voltage 52 is gradually increased until the arc detection circuitry determines that an arc is no longer present (e.g., the lens transitions to the light state), at which point the sensitivity voltage 52 is equal to an optical voltage (V_OPT) 56. Essentially, the optical voltage 56 is the voltage output by the optical sensing circuitry corresponding to the intensity of light detected. In one embodiment, an additional hysteresis (V_HYS) 58 may be added to the sensitivity voltage 52 once the optical voltage 56 is reached to reduce lens flickering due to slight variations in ambient lighting. The hysteresis voltage 58 may be, for example, a preset value, a percentage of the optical voltage 56, a user-adjustable value, or any appropriate value which is added to the optical voltage 56 to reduce the instance of lens flicker during use of the helmet assembly 20. The resultant threshold voltage 52 may be determined by the following equation:

V_SENS=V_OPT+V_HYS. (1)

In another embodiment, the sensitivity voltage 52 may be initially set to a maximum value. The voltage 52 is then gradually decreased until the arc detection circuitry determines that an arc is present (i.e., the sensitivity voltage 52 becomes less than the optical voltage 56, and the lens transitions from light to dark). The hysteresis voltage 58 may then be subtracted from the sensitivity voltage 52 to eliminate flickering. In a further embodiment, the sensitivity voltage 52 may be determined utilizing Equation 1 by digitally reading the optical voltage 56 from the optical sensing circuit and adding the hysteresis voltage 58.

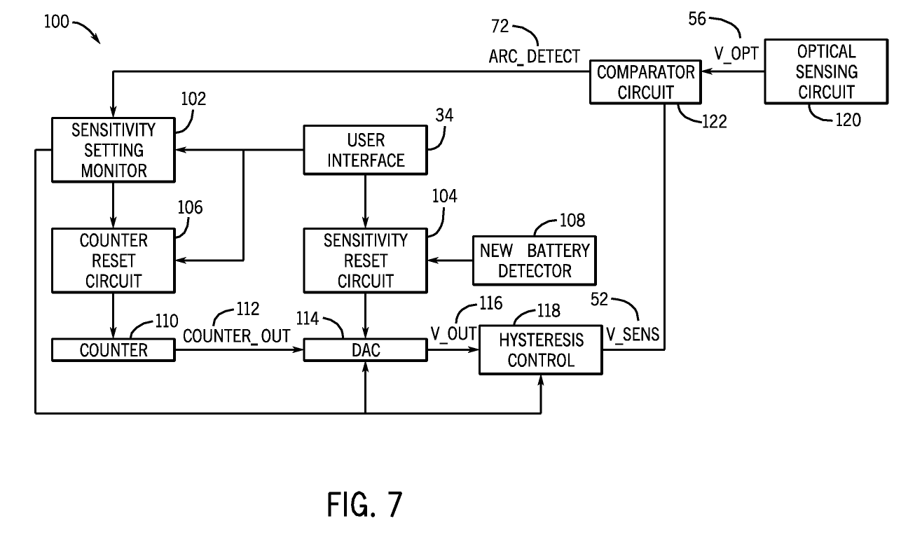

As discussed above, automatic sensitivity adjustment may be achieved via a digital or analog system. FIG. 5 is an exemplary embodiment of a block diagram illustrating a digital automatic sensitivity setting system 60. The system 60 includes the automatic adjustment interface 34 configured to initiate the automatic sensitivity adjustment sequence 50 (FIG. 4). The automatic adjustment interface 34 may include a dedicated button, a power and/or reset button, the microphone 36, a display, or any other human interface device. In one embodiment, the automatic sensitivity adjustment sequence 50 may be activated through a menu-based system navigated with menu control buttons. In another embodiment, the automatic sensitivity adjustment sequence 50 may be initiated as part of a power-up or start-up sequence.

A microprocessor 62 may receive signals from the automatic adjustment interface 34. The microprocessor 62 operates as the control center to the automatic sensitivity-setting process. In addition, the microprocessor 62 may control other functions of the helmet assembly 20, such as, for example, shade control, delay control, lens darkening (state) control, power management, temperature sensing, and so forth. The microprocessor 62 may be signaled by the automatic adjustment interface 34 to initiate the automatic sensitivity-setting process, at which point the microprocessor 62 digitally communicates with a digital-to-analog converter (DAC) 64 to produce a value for the sensitivity voltage 52. That is, the DAC 64 may convert a digital signal (D) 66 supplied by the microprocessor 62 to the analog sensitivity voltage 52. In one embodiment, as illustrated in FIG. 4, the initial digital signal 66 may correspond to an initial sensitivity voltage 52 at or near zero, and the microprocessor 62 may send increasing digital signals 66 to the DAC 64 to gradually increase the voltage 52 over time. The microprocessor 62 may communicate with the DAC 64 via any suitable communication protocol, such as, for example, parallel digital lines, serial, I²C, and so forth. In one embodiment, the DAC 64 may be a voltage divider circuit having a digital potentiometer. It may be desirable to utilize a 10-bit or higher DAC 64 to achieve adequate resolution.

As described above, the sensitivity voltage 52 may be compared to the optical voltage 56 to determine the optimal sensitivity setting. Accordingly, the optical voltage 56 may be determined by an optical sensing circuit 68. The optical sensing circuit 68 may receive input from the optical sensors 38 (FIG. 2) and output the optical voltage 56. The optical voltage 56 may be directly proportional to the magnitude of the optical energy sensed by the optical sensors 38. In one embodiment, the optical sensing circuit 68 may contain an amplification stage having linear or non-linear gain. In addition, the circuit 68 may contain a voltage biasing stage to create a voltage offset.

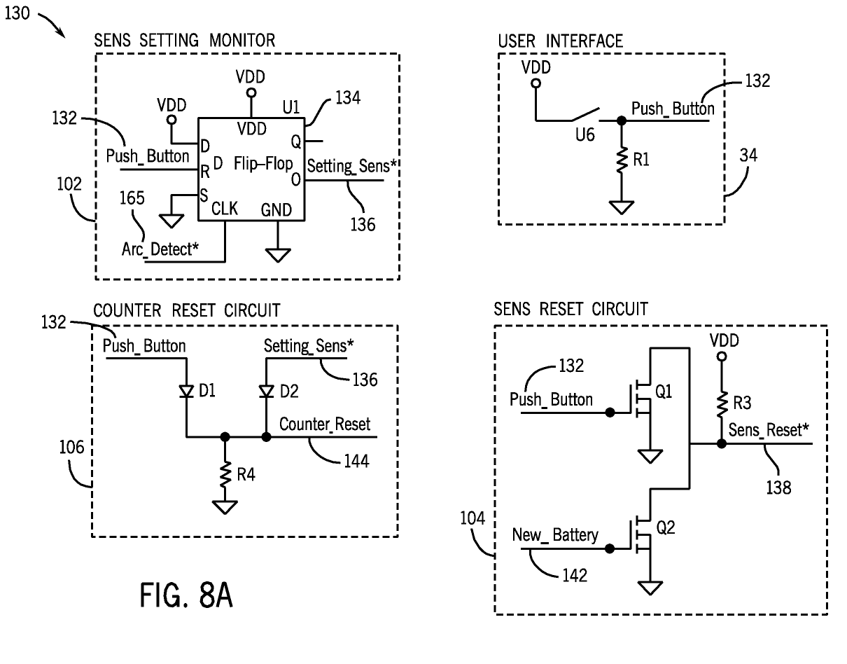

The sensitivity voltage 52 and the optical voltage 56 may be compared at a comparator circuit 70. The comparator circuit 70 may then output a digital arc detect signal (ARC_DETECT) 72 based on the sensitivity voltage 52 and the optical voltage 56. That is, the arc detect signal 72 is set to “high” if the sensitivity voltage 52 is less than the optical voltage 56 and “low” if the sensitivity voltage 52 is greater than the optical voltage 56. In an exemplary embodiment of a binary system, the digital “high” signal 72 may be equal to 1, while the digital “low” signal 72 is equal to 0, or vice versa. The arc detect signal 72 may also be utilized during operation of the helmet assembly 22 (FIG. 1) to determine if the lens assembly 24 should be in the clear or dark state. That is, when the signal 72 is “high,” the lens is in the dark state, and when the signal 72 is “low,” the lens is in the clear state. The microprocessor 62 may interpret the arc detect signal 72 to determine if the sensitivity-setting sequence may be terminated, as illustrated by an exemplary embodiment of a digital automatic sensitivity-setting process 80 in FIG. 6.

The exemplary process 80 illustrated in FIG. 6 includes an exemplary embodiment of a digital sensitivity adjustment sequence 82, which may be initiated via the automatic adjustment interface 34 (FIGS. 2 and 5). In some embodiments, before the sequence 82 is initiated, upon power being applied to the digital sensitivity setting system 60 (block 84), the digital signal 66 may be set to zero and communicated from the microprocessor 62 to the DAC 64 (block 86). The sensitivity voltage 52 is therefore set to 0 V, which is less than the optical voltage 56 except in a completely dark environment. The resulting arc detect signal 72 is high, and the corresponding lens state is dark, thereby reminding the operator 18 to initiate the adjustment sequence 82.

The sensitivity adjustment sequence 82 may begin with the microprocessor 62 monitoring for an initiation signal from the automatic adjustment interface 34 (e.g., the push of a button, a voice command, etc.) (block 88). When the initiation signal is received, the microprocessor 62 may set the digital signal 66 to a predetermined minimum value. In one embodiment, the minimum value may be zero. In another embodiment, the minimum value may be such that upon conversion in the DAC 64 (block 90), the resulting sensitivity voltage 52 is at or only slightly less than a minimum optical voltage. That is, the optical sensing circuit 68 (FIG. 5) may be configured such that when no light is detected by the sensors 38 (FIG. 2), the optical voltage 56 is some voltage greater than zero. The sensitivity voltage 52 may then be initialized at or slightly less than the minimum optical voltage to enable faster optimization of the sensitivity adjustment sequence 82.

The sensitivity voltage 52 is then compared to the optical voltage 56 at the comparator circuit 70 (FIG. 5) to produce the arc detect signal 72 (block 92). After a preset delay to allow the comparison operation to complete, the microprocessor 62 determines if the arc detect signal 72 is low (block 94). If the signal 72 is not low, the microprocessor 62 increments the digital signal 66 by a value denoted as AD (block 96). The resulting digital signal 66 is again converted via the DAC 64 (block 90), and the sensitivity voltage 52 is again compared to the optical voltage 56. If, on the other hand, the signal 72 is low, the microprocessor 62 may add a hysteresis value denoted as H, corresponding to the hysteresis voltage 56 (FIG. 5), to the digital signal 66 (block 98). The digital signal 66 is again sent to the DAC 64, where the sensitivity voltage 52 is generated (block 86). In addition, in response to the “low” arc detect signal 72, the lens may transition to the clear state, thereby indicating to the operator 18 (FIG. 1) that the sensitivity adjustment sequence 82 is complete. The sensitivity voltage 52 may remain at the last value until the operator 18 again initiates the sensitivity adjustment sequence 82.

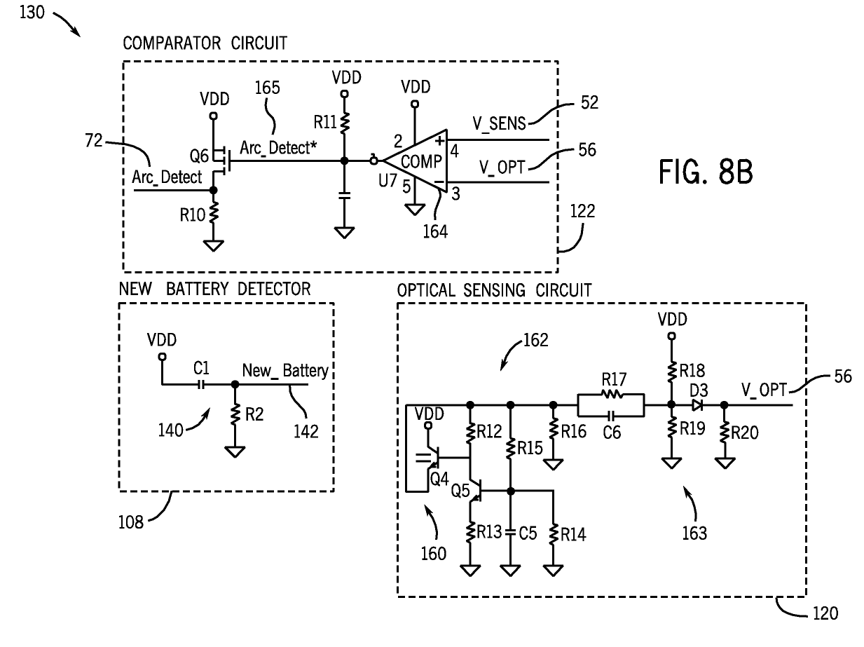

Turning to FIG. 7, a block diagram illustrates an exemplary embodiment of an analog automatic sensitivity setting system 100. The system 100 includes the automatic adjustment interface 34 configured to initiate the automatic sensitivity adjustment sequence. As described above, the automatic adjustment interface 34 may include a dedicated button, a power and/or reset button, the microphone 36, a display, or any other human interface device.

The automatic adjustment interface 34 may be coupled to a sensitivity setting monitor 102, a sensitivity reset circuit 104, and a counter reset circuit 106 such that triggering the sensitivity-setting process at the automatic adjustment interface 34 sends signals to the monitor 102 and the circuits 104 and 106. The sensitivity setting monitor 102 effectively indicates if the automatic sensitivity setting process is under way. The sensitivity reset circuit 104 prepares the system 100 for determining the correct sensitivity voltage 52 by initially resetting the sensitivity voltage 52 to ground. In addition to the automatic adjustment interface 34 sending a signal to the circuit 104, a new battery detector 108 coupled to the sensitivity reset circuit 104 also triggers the circuit 104 to set the sensitivity voltage 52 to ground, thereby reminding the operator 18 (FIG. 1) to reset the sensitivity settings upon replacement of the battery. The counter reset circuit 106 resets a counter 110 to output all zeroes, as described in more detail below.

The counter 110 sends a digital signal 112 (COUNTER-OUT) to a digital-to-analog converter (DAC) 114. That is, the counter 110 counts, for example, from 0 to 2¹⁰−1 (i.e., 1023), and outputs the counter signal 112 to the DAC 114. The DAC 114 converts this digital signal 112 into an analog signal, such as an output voltage (V_OUT) 116. A hysteresis control 118 then adds a hysteresis value to the output voltage 116 to generate the sensitivity voltage 52. The sensitivity voltage 52 and the optical voltage 56 from an optical sensing circuit 120 are compared in a comparator circuit 122. The optical sensing circuit 120 and the comparator circuit 122 may be similar to the circuit 68 and the comparator circuit 70 employed in the digital automatic sensitivity setting system 60 (FIG. 5).

The arc detect signal 72 may be output from the comparator circuit 122 to the sensitivity setting monitor 102, again indicating if the sensitivity voltage 52 is higher or lower than the optical voltage 56. That is, the arc detect signal 72 may be “high” if the sensitivity voltage 52 is less than the optical voltage 56 and “low” if the sensitivity voltage 52 is greater than the optical voltage 56. When the arc detect signal 72 transitions from “low” to “high,” the sensitivity setting monitor 102 is signaled to complete the automatic sensitivity setting process.

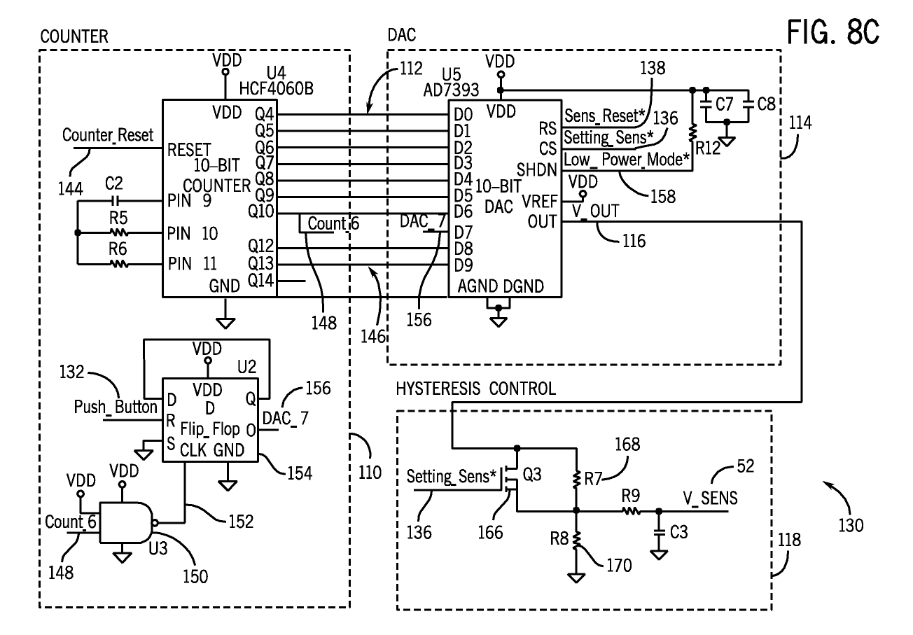

Exemplary embodiments of the components of FIG. 7 are described in more detail in a circuit diagram 130 in FIG. 8. In the circuit diagram 130, a signal followed by an asterisk (*) indicates that the Boolean equivalent of the signal value (“low”=False, “high”=True) indicates the inverse (or “not”) of the signal name. In the illustrated embodiment, the automatic adjustment interface 34 may be a push button such that while the push button is depressed, a digital signal (PUSH_BUTTON) 132 is set to “high.” By setting the PUSH_BUTTON signal 132 to “high,” the sensor reset circuit 104 may reset a D flip-flop 134 in the sensor setting monitor 102, thereby setting an output signal (SETTING_SENS*) 136 to “low.” While the SETTING_SENS* signal 138 is “low” the automatic sensitivity setting process will proceed. In addition, the “high” PUSH_BUTTON signal 132 may set an output signal (SENS_RESET*) 138 from the sensor reset circuit 104 to “low.” Similarly, a high-pass filter 140 in the new battery detector 108 may cause a signal (NEW_BATTERY) 142 from the new battery detector 108 to the sensor reset circuit 104 to switch to “high,” also setting the SENS_RESET* signal 138 to “low.” When the SENS_RESET* signal 138 is low, the output voltage 116 from the DAC 114 is reset to ground, and the lens darkens. The operator 18 (FIG. 1) is therefore reminded to reinitiate the automatic sensitivity setting process when a new battery is inserted.

The “high” PUSH_BUTTON signal 132 may also maintain an output signal (COUNTER_RESET) 144 from the counter reset circuit 106 at “high,” which in turn resets the COUNTER_OUT signal 112 to all zeroes. Upon releasing the push button, the COUNTER_RESET signal 144 may be set to “low,” taking the counter 110 out of reset mode. In the illustrated embodiment, the counter 110 is a 10-bit counter, therefore it counts from 0 to 2¹⁰−1 (i.e., 1023) via 10 digital output lines 146. The exemplary counter 110 has output lines Q4-Q10 and Q12-Q14, with no output Q11. Accordingly, the output from Q10 has a quarter of the period of the output from Q12. In order to create the intermediate bit between Q10 and Q12, an output signal (COUNT_{—}6) 148 from Q10 may be inverted at an inverter 150 and then used as a clocking input 152 to a D flip-flop 154. The D flip-flop 154 outputs a digital signal (DAC_{—}7) 156 which becomes the missing bit input into the DAC 114.

When the DAC 114 is not in reset mode and the SETTING_SENS* signal 136 is “low,” the analog output V_OUT 116 reflects the changing 10-bit input value COUNTER_OUT 112 according to the following equation:

$$V\_OUT = VDD \times ( \frac{COUNTER\_OUT}{1024} ) . \qquad ( 2 )$$

where VDD is the power supply voltage. Accordingly, as the counter 110 counts from 0 to 1023, the output voltage V_OUT 116 increases, resulting in an increasing value of V_SENS 52 from the hysteresis control 118. An additional input signal (LOW_POWER_MODE*) 158 may place the DAC 114 in a low power consumption state when the signal 158 is “low.” The LOW_POWER_MODE* signal 158 may be controlled by other circuitry (not shown) related to other lens operations. While in the low power consumption state, the DAC 114 may maintain the current sensitivity value V_OUT 116 in memory but temporarily ground the V_OUT 116. When the LOW_POWER_MODE* signal 158 transitions to “high,” the V_OUT 116 reverts to the previously stored value.

As described above, the sensitivity voltage 52 is compared to the optical voltage 56 at the comparator circuit 122. The optical sensing circuit 120 may include a phototransistor 160, an amplification stage 162, and a voltage biasing stage 163. In the illustrated embodiment, the V_OPT 56 may be directly related to the intensity of light impacting the phototransistor 160. The voltages 52 and 56 may be compared at a comparator 164 in the comparator circuit 122. An ARC_DETECT* signal 165 may be output from the comparator 164 and inverted to output the ARC_DETECT signal 72 from the comparator circuit 122. Again, the ARC_DETECT signal 72 may be “low” if the V_OPT 56 is greater than the V_SENS 52. When the V_SENS 52 exceeds the V_OPT 56, the ARC_DETECT signal 72 may transition to “high,” at which time the sensitivity setting monitor 102 is signaled to complete the automatic sensitivity setting process.

When the ARC_DETECT signal 72 clocks into the D flip-flop 134 of the sensitivity setting monitor 102 at “high,” this changes the SETTING_SENS* signal 136 to “high,” indicating that the automatic sensitivity setting process is complete. After the SETTING_SENS* signal 136 is set to “high,” it remains “high” until the automatic sensitivity setting process is reinitiated. When the SETTING_SENS* signal 136 is changed to “high,” the value of the COUNTER_OUT signal 112 from the counter 110 is latched into the memory of the DAC 114, forcing the V_OUT 116 from the DAC 114 to remain constant at a value reflecting the latched counter value.

The hysteresis control 118 then adds a hysteresis to the V_OUT 116. The “high” SETTING_SENS* signal 136 turns off a hysteric control transistor 166, which had been shorting out a voltage divider resistor 168. The V_OUT 116 is then increased by the hysteresis to generate the sensitivity voltage V_SENS 52 according to the following equation:

$$V\_SENS = V\_OUT \times \frac{R 7}{R 7 + R 8} , \qquad ( 3 )$$

where R7 and R8 are the voltage divider resistors 168 and 170. In addition, the “high” SETTING_SENS* signal 136 puts the counter 110 into reset mode, thereby reducing power consumption when the sensitivity is not being automatically set.

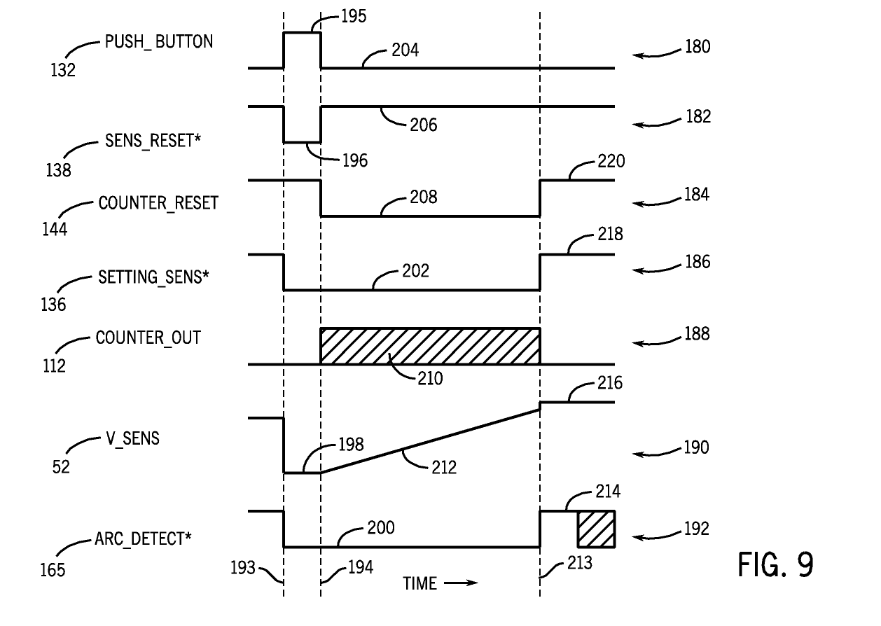

FIG. 9 illustrates exemplary embodiments of timing diagrams for various signals associated with the exemplary analog automatic sensitivity setting system 100 of FIGS. 7-8. The PUSH_BUTTON signal 132 is represented in a timing diagram 180, the SENS_RESET* signal 138 is represented in a timing diagram 182, the COUNTER_RESET signal 144 is represented in a timing diagram 184, the SETTING_SENS* signal 136 is represented in a timing diagram 186, the COUNTER_OUT value 112 is represented in a timing diagram 188, the V_SENS signal 52 is represented in a timing diagram 190, and the ARC_DETECT* signal 165 is represented in a timing diagram 192. The digital signals PUSH_BUTTON 132, SETTING_SENS* 136, SENS_RESET* 138, COUNTER_RESET 144, and ARC_DETECT* 165 may be either “high” or “low,” and the timing diagrams 180, 186, 182, 184, and 192, respectively, reflect this binary function. That is, a “high” signal is represented by a higher section of the diagram, whereas a “low” signal is represented by a lower section of the diagram.

As described above, the operator 18 (FIG. 1) may initiate the analog automatic sensitivity setting process by activating the user interface 34 (e.g., pushing the button) at a time 193. Although the timing diagrams 180, 182, 186, 190, and 192 show events occurring simultaneously, it should be understood that these events may actually occur sequentially within a short time period. For example, minor time deviations may be due to the time it takes signals to be relayed from one component of the analog automatic sensitivity setting system 100 to another.

While the user interface 34 is activated (e.g., while the button is depressed) from the time 193 to a time 194, the PUSH_BUTTON signal 132 is “high,” as indicated by a high section 195 of the timing diagram 180. The “high” PUSH_BUTTON signal 132 sets the SENS_RESET* signal 138 to “low,” as indicated by a low section 196 of the timing diagram 182. The “low” SENS_RESET* signal 138 in turn resets the output of the DAC 114 (FIGS. 7 and 8), thereby setting the V_SENS 152 to ground, as indicated by a flat section 198 of the timing diagram 190. When the V_SENS 52 goes to ground, the ARC_DETECT* signal 165, which results from the comparison of the V_SENS 52 with the V_OPT 56 (FIGS. 7 and 8), is set to “low”, as indicated by a “low” section 200 of the timing diagram 192. The “high” PUSH_BUTTON signal 132 also sets the SETTING_SENS* signal 136 to “low,” as indicated by a low section 202 of the timing diagram 186. While the SETTING_SENS* signal 136 is “low” and the SENS_RESET* signal 138 is “high,” the analog sensitivity setting process continues.

When the user interface 134 is no longer activated (e.g., when the operator 18 releases the button) at the time 194, the PUSH_BUTTON signal 132 returns to “low,” as indicated by a low section 204 of the timing diagram 180, and the SENS_RESET* signal 138 returns to “high,” as indicated by a high section 206 of the timing diagram 182. The COUNTER_RESET signal 144 is also set to “low” at the time 194, as indicated by a low section 208 of the timing diagram 184. When the COUNTER_RESET signal 144 converts to “low,” the counter 110 (FIGS. 7 and 8) begins to count, and the 10-bit digital COUNTER_OUT value 112 increases, as indicated by a gray section 210 on the timing diagram 188. Because the V_SENS 52 is an analog conversion of the digital COUNTER_OUT value 112 input into the DAC 114, the V_SENS 52 increases along with the COUNTER_OUT value 112, as indicated by a sloping section 212 on the timing diagram 190.

When the V_SENS 52 is greater than the V_OPT 56 at a time 213, the ARC_DETECT* signal 165 converts to “high,” as indicated by a high section 214 on the timing diagram 192. The V_SENS 52 at the time 213 is increased by the hysteresis then maintained at a set value, as indicated by a flat section 216 on the timing diagram 190. The “high” ARC_DETECT* signal 165 is input into the sensitivity setting monitor 102 (FIGS. 7 and 8), and the output SETTING_SENS* signal 136 converts to “high,” as indicated by a high section 218 on the timing diagram 186. When the SETTING_SENS* signal 136 goes “high,” the analog automatic sensitivity setting process stops. Accordingly, the COUNTER_RESET signal 144 returns to “high,” as indicated by a high section 220 on the timing diagram 184, thereby resetting the counter 110 such that the COUNTER_OUT value 112 is again zero.

Benefits of the systems and methods described herein may include ease of use, reduced costs, and improved safety. For example, setting sensitivity automatically may be easier for new operators to learn and may take less time. In embodiments in which the lens is powered on or started up, the automatic sensitivity setting process may be integrated into the power-up or start-up sequence. A separate sensitivity knob may be replaced by a lower-cost button and/or integrated with an existing button, thereby eliminating the need for a dedicated sensitivity knob. Furthermore, the automatic sensitivity setting process may be more precise than a manual sensitivity setting process. In some instances, such as when using a low-amperage TIG welding process, it may be desirable to activate the lens's auto-darkening function with only a small change in light. When the sensitivity is based on signals from optical sensors rather than the operator's eyes, the sensitivity setting may be closer to the ambient lighting, thereby increasing the sensitivity of the lens to additional light without inducing flicker.

While only certain features of the invention have been illustrated and described herein, many modifications and changes will occur to those skilled in the art. It is, therefore, to be understood that the appended claims are intended to cover all such modifications and changes as fall within the true spirit of the invention.

What is claimed is:

1. A welding helmet, comprising:
an auto-darkening lens, comprising:
one or more optical sensors configured to sense optical energy;
optical sensing circuitry configured to convert the sensed optical energy into an electrical voltage;
sensitivity circuitry configured to automatically adjust a threshold sensitivity voltage based on the electrical voltage; and
a user input configured to initiate automatic adjustment of the threshold sensitivity voltage.

2. The helmet of claim 1, wherein the threshold sensitivity voltage is adjustable only when initiated by the user input.

3. The helmet of claim 1, wherein the user input comprises a button, a switch, a slider, or a knob.

4. The helmet of claim 1, wherein the user input comprises a power or reset control of the auto-darkening lens.

5. The helmet of claim 1, wherein the user input is electrically connected to the auto-darkening lens.

6. The helmet of claim 1, wherein the user input is wirelessly connected to the auto-darkening lens.

7. The helmet of claim 1, wherein the user input is accessible to a user while wearing the welding helmet.

8. The helmet of claim 1, comprising a comparator configured to compare the electrical voltage to the threshold sensitivity voltage and output a digital signal indicative of the comparison.

9. The helmet of claim 8, wherein the sensitivity circuitry is configured to adjust the threshold sensitivity voltage at least until the digital signal from the comparator changes.

10. The helmet of claim 9, wherein the sensitivity circuitry is configured to adjust the threshold sensitivity voltage by an amount of hysteresis after the digital signal from the comparator changes.

11. The helmet of claim 10, wherein the amount of hysteresis is adjustable.

12. The helmet of claim 1, wherein the sensitivity circuitry comprises a microprocessor.

13. The helmet of claim 12, comprising an analog-to-digital converter configured to convert the electrical voltage from the optical sensing circuitry to a digital signal, wherein the microprocessor is configured to adjust the threshold sensitivity voltage based on the digital signal.

14. The helmet of claim 1, wherein the sensitivity circuitry comprises an integrated circuit counter.

15. The helmet of claim 1, comprising a device configured to alert a user of the automatic adjustment to the threshold sensitivity voltage.

16. The helmet of claim 1, comprising override circuitry configured to enable override of the automatic adjustment to the threshold sensitivity voltage.

17. The helmet of claim 16, wherein the override circuitry enables manual adjustment of the threshold sensitivity voltage relative to the automatically adjusted threshold sensitivity voltage.

18. A method for automatically adjusting sensitivity in an auto-darkening lens, comprising:
detecting ambient light intensity;
converting the ambient light intensity to an optical voltage;
comparing the optical voltage to a sensitivity voltage and outputting a digital signal based on the comparison; and
automatically adjusting the sensitivity voltage stepwise until the digital signal changes value.

19. The method of claim 18, comprising adjusting the threshold sensitivity voltage by an amount of hysteresis after the digital signal from the comparator changes.

20. The method of claim 18, comprising forcing the lens to a darkened state upon first applying power to the lens, restarting the lens, resetting the lens, and/or reinitializing the lens.

21. The method of claim 18, comprising forcing the lens to a darkened state while the sensitivity threshold is being adjusted.

22. An auto-darkening lens, comprising:
a user interface configured to initiate an automatic sensitivity setting process;
sensitivity setting circuitry configured to set a sensitivity voltage to an initial value upon initiation of the automatic sensitivity setting process;
one or more optical sensors configured to detect light intensity;
an optical sensing circuit configured to convert the light intensity to an optical voltage; and
a comparator configured to compare the sensitivity voltage to the optical voltage and output a digital signal indicative of the comparison;
wherein the sensitivity setting circuitry is configured to alter the sensitivity voltage until the digital signal from the comparator changes.

23. The lens of claim 22, wherein the user interface comprises a button, a switch, a slider, or a knob.

24. The lens of claim 22, wherein the sensitivity setting circuitry comprises a microprocessor.

25. The lens of claim 22, wherein the sensitivity setting circuitry comprises an integrated circuit counter.

26. The lens of claim 22, wherein the sensitivity setting circuitry is configured to adjust the sensitivity voltage with hysteresis after the digital signal from the comparator changes.
